(19) United States
(12) Patent Application Publication
(10) Pub. No.: US 2002/0065442 A1
(43) Pub. Date: May 30, 2002

(54) Radial reactor loading

(75) Inventors: David L. Williams, Louisville, KY (US); Andrzej Rokicki, Prospect, KY (US); Dennis J. Smith, Louisville, KY (US); Kyle Mankin, Prospect, KY (US)

Correspondence Address: Scott R. Cox, Suite 2200, 400 West Market St., Louisville, KY, 40202, US

(73) Assignee: Sud-Chemie Inc., Louisville, KY

(21) Appl. No.: 09/923,658

(22) Filed: Aug. 7, 2001

Related U.S. Application Data
(63) Continuation-in-part of application No. 09/727,036, filed on Nov. 30, 2000.

(51) Int. Cl. B01J 8/02; C07C 5/333; C07C 5/327

(52) U.S. Cl. 585/440; 422/218; 422/211; 585/444

(57) ABSTRACT

A radial reactor for utilization for catalytic reactions of gaseous or liquid feed streams including a vertical, annular catalyst bed, wherein the material contained within the catalyst bed includes an active catalyst material, contained within an outer ring-shaped, vertical layer of the catalyst bed, and an inert material, contained within an inner ring-shaped, vertical layer of the catalyst bed.

### CROSS REFERENCE TO RELATED APPLICATIONS

None

### BACKGROUND 0F INVENTION

1. Field of Invention

The field of art to which this invention pertains is to the composition of a catalyst bed contained in a radial reactor, wherein the catalyst bed includes catalyst material and an inert material. More particularly, this invention relates to a catalyst bed for a radial reactor for dehydrogenation reactions, wherein the catalyst bed is divided into an inner and an outer ring-shaped, vertical layer, wherein catalyst material is contained in the outer vertical layer and an inert material is contained in the inner, vertical layer.

2. Description of Related Art.

Radial reactors are utilized for a number of different types of catalytic reactions. For example, radial reactors are utilized in ammonia synthesis plants as disclosed in U.S. Pat. Nos. 4,880,603 and 5,250,270.

Various designs for radial reactors and the flow pattern of feed streams within those radial reactors have been disclosed in a number of patents owned by Ammonia Casale S. A. The reactions that occur in those radial reactors are generally heterogenous synthesis reactions, such as ammonia synthesis or methanol synthesis. The reactors and their catalyst beds are designed to encourage various patterns of flow of the feed stream through the catalyst beds within the radial reactors. These radial reactors are disclosed, for example, in U.S. Pat. Nos. 5,756,048, 5,006,316, 4,963,338, 4,952,375, 4,904,453, 4,755,362, 4,769,220, 4,405,562 and 4,372,920.

A common commercial chemical process where radial reactors are utilized is the dehydrogenation of hydrocarbons. The process for the dehydrogenation of hydrocarbons is well described in the prior art, whereby both acyclic and aromatic hydrocarbons are converted to correspondingly less saturated hydrocarbon products. One of the best known of these dehydrogenation processes is the conversion of alkyl aromatics, particularly ethylbenzene to styrene. In this process ethylbenzene is reacted at an elevated temperature over a dehydrogenation catalyst, such as iron oxide to form styrene. A process for the dehydrogenation of ethylbenzene to styrene and catalysts used for that reaction are disclosed in U.S. Pat. No. 6,096,937.

The commercial process for the conversion of ethylbenzene to styrene is normally conducted in a series of radial, adiabatic reactors rather than in a single reactor. Radial reactors utilized for the production of styrene are disclosed in U.S. Pat. Nos. 3,475,508, 3,515,763 and 3,918,918. These radial reactors generally are elongated, cylindrical, vertical structures which may be very large, ranging in diameter from 5 to 20 feet (150 to 610 cm) or more and in length from 5 to about 100 feet (150 to 3050 cm) or more. Examples of radial reactor designs are disclosed in the drawings associated with JP 49039971 and 49039972. A process and apparatus for the conversion of ethylbenzene to styrene in a radial reactor are also disclosed in U.S. Pat. Nos. 5,358,698 and 4,039,601.

It has been recognized that a reactor system containing multiple radial reactors may produce a higher degree of conversion of the hydrocarbon and may have greater product yield than is exhibited by use of a single radial reactor. Thus, sometimes three or more radial reactors are often arranged in a serial flow orientation with reheat means, which may be located both within and between the reactors, to add heat to the reaction.

Conventional radial reactors contain an inlet located in the center of the radial reactor assembly. Catalysts for the reaction are placed within a bed or beds in that reactor assembly, generally occupying a ring-shaped, vertical space, which is located outside of a central core of the reactor. The feed stream enters the reactor through the inlet and then flows radially outward through the catalyst material contained in the vertical catalyst bed to an open, annular space, which is formed outside of the catalyst bed but within the reactor assembly. Ultimately the feed stream flows to an outlet as shown, for example, in U.S. Pat. No. 3,898,049.

Modifications to this basic design have been considered, such as are disclosed in U.S. Pat. No. 5,358,698. The goal of such modified arrangements is to enhance certain performance and operating characteristics of the catalyst, such as selectivity and activity. “Selectivity” is the ability of a catalyst to selectively produce higher levels of the desired product and lower levels of undesired products. For example, in the conversion of ethylbenzene to styrene, higher selectivity results in higher levels of styrene and lower levels of various by-products. “Activity” is the ability of the catalyst to convert a certain percentage of the ethylbenzene during each pass of the feedstock over the catalyst. Higher activity means a higher percentage of the ethylbenzene is converted.

The conversion of ethylbenzene to styrene is an endothermic reaction which requires the addition of heat to the process to maintain an appropriate level of activity. The rate of flow of the ethylbenzene must be controlled as it passes across the catalyst bed to maintain acceptable selectivity and activity. Thus, the liquid hourly space velocity (LHSV) of the feed stream through the catalyst bed of the reactor must be regulated. The LHSV is generally maintained at a given rate regardless of the thickness of the catalyst bed in the reactor assembly.

Catalyst material loses selectivity and activity over time. Multiple reactors have been used to overcome this problem. One proposed design for such reactors is disclosed in U.S. Pat. No. 6,096,937.

There are two entirely different recognized types of ethylbenzene dehydrogenation reaction. The conventional dehydrogenation reaction (nonoxidative dehydrogenation) occurs over an iron oxide catalyst and requires that oxygen not be present in the feed stream.

The less utilized, second type of dehydrogenation reaction is oxidative dehydrogenation where the feed stream contains ethylbenzene and a source of oxygen. In this second process, the feed stream is passed over an oxidative dehydrogenation catalyst, where an oxidation reaction occurs to generate heat. The reaction is generally as follows:

C₆H₅CH₂CH₃+1/2O₂→C₆H₅CH=CH_{2+H}₂0

Oxidative dehydrogenation is exothermic and irreversible. An oxidative dehydrogenation reaction is disclosed for example in U.S. Pat. Nos. 5,510,553 and 4,777,319. This process is also discussed in Kirk-Othmer, Encyclopedia of Chemical Technologies, Volume 22, page 978 (1996). The process of “oxidative” dehydrogenation has not attained commercial acceptance for various reasons, such as the safety of the oxygen mixing step, potential damage to the dehydrogenation catalyst by oxidation and the possibility of contaminating the styrene product with oxygenates.

In addition, UOP has disclosed a process for oxidatively reheating a reaction mixture including ethylbenzene using a radial reactor system containing two oxidation catalyst beds utilized in combination with three conventional dehydrogenation catalyst beds in U.S. Pat. No. 5,043,500. In the process of this patent an oxidation catalyst bed (64) is physically located in a vertically layered bed next to a dehydrogenation catalyst (66) also contained within the reactor. UOP has disclosed the use of one, two or more oxidation catalyst beds. See also Kirk-Othmer, Encyclopedia of Chemical Technologies, Volume 22, pages 978-980 (1996).

Conventional “nonoxidative” dehydrogenation reactions generally use radial reactors but conventionally utilize only a single dehydrogenation catalyst, such as a conventional iron oxide catalyst containing a small amount of potassium and chrome as disclosed, for example, in U.S. Pat. Nos. 2,866,790 and 2,866,791. Various catalysts for nonoxidative dehydrogenation are also disclosed in U.S. Pat. No. 6,191,065, the contents of which are incorporated herein by reference.

Conventional nonoxidative dehydrogenation catalysts for use in the conversion of ethylbenzene to styrene in reactors gradually deactivate over time during normal use, causing a reduction in ethylbenzene conversion. Further, there are limitations on the activity and selectivity of conventional dehydrogenation catalysts. Other problems that may occur when conventional nonoxidative catalysts are utilized in a conventional radial reactor include reduced steam to oil stability, shortened life span, increased pressure drop, loss of stability at reaction pressures and increased contamination.

In radial reactors used for ethylbenzene dehydrogenation, the gas feed flows radially from the central core of the reactor assembly through catalyst material contained in a ring-shaped, vertical catalyst bed contained within the radial reactor. However, because many such catalytic reactions, including nonoxidative dehydrogenation of ethylbenzene, are temperature sensitive, the volume of the catalyst material within the catalyst bed that actually catalyzes the feed stream is often limited. For example, in nonoxidative dehydrogenation radial reactors for the dehydrogenation of ethylbenzene, only the first 4 inches (10 cm) to 15 inches (40 cm) or so of thickness of the catalyst material contained in the ring-shaped, vertical layer of the catalyst bed effectively dehydrogenates the ethylbenzene feed stream. Because the reaction is adiabatic, by the time the ethylbenzene feed stream has passed 18 inches (46 cm) or so through the catalyst bed, the temperature of the feed stream has dropped to such an extent that the activity of the reaction is diminished dramatically or even extinguished. Further, when the temperature of the ethylbenzene feed stream drops as it passes through a thick catalyst bed, a higher percentage of undesired by-products are produced. In addition, the greater the thickness of the catalyst bed, the greater the pressure drop as the feed stream passes through the catalyst bed.

Notwithstanding these reductions in the performance of the catalyst material in the thick catalyst beds contained in large diameter radial reactors, it has become conventional to build shorter reactor assemblies with thicker catalyst beds instead of building taller radial reactors with thinner catalyst beds because of the high cost in building both the support structure for the reactors and the radial reactors themselves. While shorter, thicker radial reactors contain the same overall quantity of catalyst material as taller, thinner radial reactors, the performance of these shorter, thicker reactors is not as efficient as when a taller, but narrower reactor is utilized.

Accordingly, it is an object of this invention to disclose a catalyst composition contained within a catalyst bed of a new or existing radial reactor assembly resulting in higher selectivity and activity of the catalyst composition than occurs with a conventional catalyst composition loading.

It is a further object of this invention to disclose a composition of catalyst material contained within a catalyst bed of a new or existing radial reactor assembly which results in a reduction in pressure drop over the pressure drop experienced when a conventional catalyst is loaded in that same radial reactor.

It is a still further object of the invention to disclose a composition of catalyst material contained within a catalyst bed of a new or existing radial reactor assembly, which reduces the percentage of undesired byproducts that are produced in comparison with the byproducts produced by a conventional catalyst loading in that same catalyst bed.

It is a further object of this invention to disclose a loading of material within a catalyst bed utilized within a new or existing radial reactor, wherein the material is loaded in an inner and an outer ring-shaped, vertical layer within the catalyst bed, wherein the inner, vertical layer is filled with an inert material and the outer, vertical layer is filled with an active catalyst material.

It is a further object of this invention to disclose a loading of material within a catalyst bed for a new or existing radial reactor utilized for the nonoxidative dehydrogenation of an alkylaromatic feed stream, wherein the material is a combination of a dehydrogenation catalyst, placed within an outer, ring-shaped, vertical layer of the catalyst bed and an inert material, placed within an inner, ring-shaped, vertical layer of the catalyst bed of the radial reactor.

It is another object of this invention to disclose a novel process for the nonoxidative dehydrogenation of an alkylaromatic feed stream in a new or existing radial reactor which includes passing the alkylaromatic feed stream through a catalyst bed in the radial reactor, wherein the catalyst bed contains a nonoxidative dehydrogenation catalyst placed within an outer, ring-shaped, vertical layer of the catalyst bed and an inert material placed within an inner, ring-shaped, vertical layer of the catalyst bed.

These and other objects can be obtained by the disclosed composition of the materials contained in catalyst beds of new or existing radial reactors and processes for the loading and utilization of those radial reactors.

### SUMMARY OF THE INVENTION

This invention is directed to a composition of material loaded into a catalyst bed of a radial reactor for catalytic reactions of gaseous or liquid feed streams, preferably for the dehydrogenation of alkyl aromatics, wherein the radial reactor comprises a conventional radial reactor assembly containing an annular, ring-shaped, vertical catalyst bed, and wherein the material loaded within the catalyst bed comprises an active catalyst material, contained within a first ring-shaped, vertical layer of the catalyst bed, and an inert material, contained within a second, ring-shaped, vertical layer of the catalyst bed. Preferably, the catalyst material is contained within an outer ring-shaped, vertical layer of the catalyst bed and the inert material is contained within an inner ring-shaped, vertical layer of the catalyst bed. Preferably the thickness of the vertical layer that holds the catalyst material is from about 4 inches (10 cm) to about 36 inches (90 cm), more preferably from about 6 inches (15 cm) to about 24 inches (60 cm), most preferably about 18 inches (46 cm).

In a preferred embodiment, the inert material comprises a material which neither promotes catalytic reactions nor interferes with the desired catalytic reaction that occurs within the radial reactor. Also, preferably, the use of the inert material in the inner, ring-shaped, vertical layer, results in no additional pressure drop of the feed stream, and most preferably, it results in a reduced pressure drop of the feed stream in comparison with the pressure drop that occurs when the catalyst bed contains only active catalyst material. In a further preferred embodiment the inert material comprises an alpha alumina or ceramic material with a size similar to that of the active catalyst material or a monolithic structure. Different sized materials can be utilized as long as there is not significant movement of either the catalyst or the inert material within the catalyst bed after loading.

This invention is also directed to a process for the nonoxidative dehydrogenation of an alkylaromatic feed stream comprising passing the alkylaromatic feed stream through a radial reactor containing a catalyst bed, wherein the catalyst bed comprises an inner, ring-shaped, vertical layer and an outer, ring-shaped, vertical layer and wherein nonoxidative dehydrogenation catalyst material is loaded within the outer, vertical layer and inert material is loaded within the inner, vertical layer.

### BRIEF DESCRIPTION OF THE DRAWING

FIG. 1 is a schematic view of a radial reactor of the invention.

FIG. 2 is a top view of the radial reactor of FIG. 1.

FIG. 3 is a cut away perspective view of the catalyst bed of the radial reactor of FIG. 1.

### DETAILED DESCRIPTION OF THE INVENTION

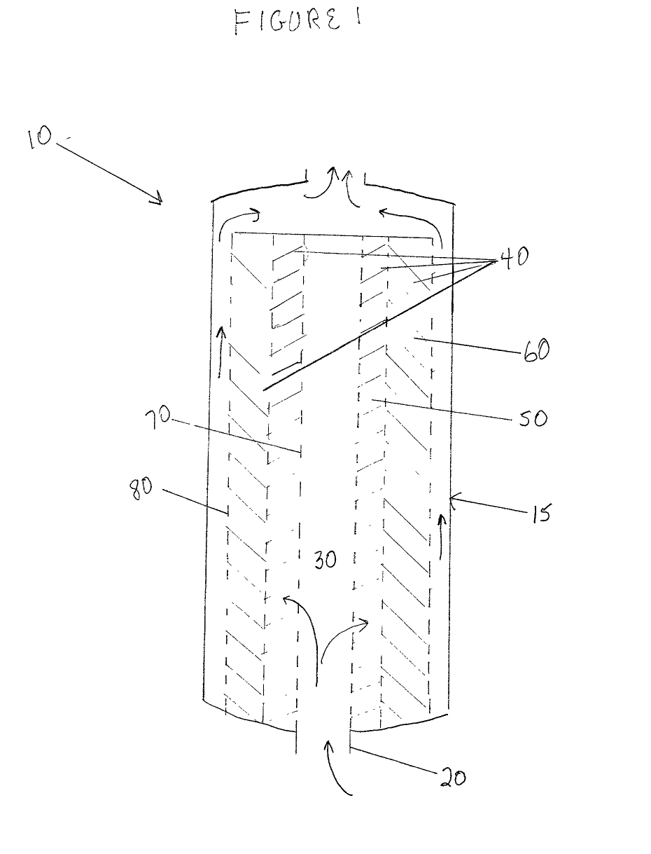

The invention is a composition for material loaded within a catalyst bed (40) within a radial reactor (10) utilized for catalytic reactions of gaseous or liquid feed streams, which reactor (10) includes a conventional radial reactor assembly (15), which contains a vertical, annular catalyst bed (40), wherein the catalyst bed is loaded with an active catalyst material (60), contained within a first ring-shaped, vertical layer of the catalyst bed (40), and an inert material (50) contained within a second ring-shaped, vertical layer of the catalyst bed (40). Referring to FIG. 1 which is a cross-sectional schematic drawing showing one embodiment of the radial reactor (10) of the invention, a feed stream is introduced through an inlet pipe (20) into the center (30) of the radial reactor (10). When the reaction is a nonoxidative dehydrogenation reaction, the hydrocarbon feed stream comprises an alkylaromatic material, preferably ethylbenzene. In this reaction the feed stream is heated by mixing it with super heated steam. The heated feed stream enters the inlet pipe (20) of the radial reactor (10) and is radially distributed through the catalyst bed (40) as shown by the arrows in FIG. 1.

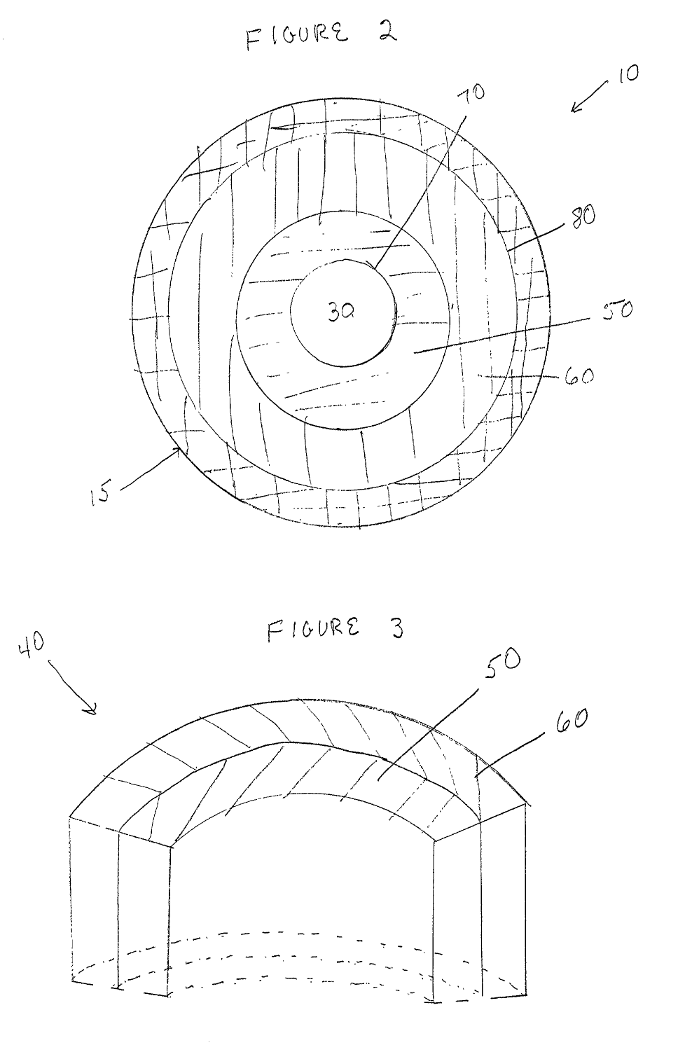

The catalyst bed (40) of the invention is comprised of two or more layers of material, preferably arranged in ring-shaped, vertical layers of material loaded in the reactor bed (40) as shown in FIGS. 1, 2 and 3. (While only a pair of vertical layers are shown in FIGS. 1, 2 and 3, three or more layers are within the scope of the invention.) When two (2) layers of material are loaded within the catalyst bed (40), the two (2) layers preferably comprise a layer of an active catalyst material (60) and a layer of inert material (50). For example, when the reaction is the nonoxidative dehydrogenation of ethylbenzene, one layer of the catalyst bed is comprised of an active catalyst (60) for that reaction and the second layer is comprised of an inert material (50), which neither promotes catalytic reactions nor interferes with the nonoxidative dehydrogenation reaction.

Each of the materials are preferably arranged in a separate vertical, annular layer within the radial reactor (10) as shown in FIG. 1. Although formal separation of the two vertical layers of material can be effected by use of a device such as a screen, such as is shown in U.S. Pat. No. 5,043,500, in a preferred embodiment the two separate layers of material (50, 60) are in intimate contact with each other within the radial reactor (10). In this preferred arrangement some mixing of the catalyst material with the inert material may occur at the boundary line between the different materials, although substantial mixing is not preferred.

The materials are retained in place within the catalyst bed (40) of the radial reactor (10) by use of an inner screen (70) and an outer screen (80), as shown in FIG. 1.

In a preferred embodiment, the catalyst bed (40) contains at least two vertical layers (50, 60) of material, wherein at least one of the layers of material is the active catalyst material (60) and at least one of the layers is the inert material (50). Many different combinations of active catalysts and inert materials are within the scope of the invention. For example, in a preferred embodiment for the non-oxidative dehydrogenation of or alkyl aromatic feed stream, a nonoxidative dehydrogenation catalyst, which has high activity is placed in the outer layer (60) of the catalyst bed (40) and an inert material, such as a ceramic or alpha alumina material, is placed within the inner layer (50) within the radial reactor (10).

By placing the active catalyst material in the outer ring-shaped, vertical layer (60) of the catalyst bed (40), there is an increase in the overall initial surface area of the catalyst material that is exposed to the hot, preheated feed stream as the feed stream begins passage through the catalyst bed (40) as shown in FIG. 3. There is an increase in the overall initial surface area of the catalyst layer because it is a further distance from the center (30) of the radial reactor (10) as shown in FIGS. 1, 2 and 3, thus presenting a greater vertical surface of catalyst material for reaction with the hot, preheated feed stream. Because the inert material does not interfere with the reaction and because a greater surface area of the catalyst material is exposed to the feed stream, there is an increase in performance of the overall catalyst bed (40) over a conventional catalyst bed, wherein the inner ring-shaped, vertical layer (50) of the catalyst bed (40) is also filled with catalytic material.

This arrangement of material in a radial reactor (10) results in a greater volume of catalytic material that is “effectively” utilized in the catalyst bed (40). In most reactions, the “effective” portion of the catalyst bed (40) that is utilized is only the first 4 inches (10 cm) to about 15 inches (40 cm) or so of the thickness of the catalyst bed (40). Because the design of the catalyst loading of the catalyst bed of the invention has shifted the effective volume of catalytic material further outward within the catalyst bed (40), as shown, for example, in FIG. 3, there is a greater overall volume of catalyst material contained within the first 4 to 15 inches (10-40 cm) of the active catalyst material using the design of the catalyst bed of the invention than would be present in the first 4 to 15 inches (10-40 cm) of a catalyst bed that did not contain inert material within an inner ring-shaped, vertical layer (50) of the catalyst bed (40).

There are other improvements which result from this combination of an inert material utilized with a catalytic material. For example, because the inert material preferably has greater physical stability than that of the catalytic material which it replaces, there is a reduction in both the initial pressure drop and the increase in pressure drop with aging of catalyst as the feed stream passes radially through the catalyst bed (40). The liquid hourly space velocity (LHSV) is also increased.

This arrangement also reduces unwanted byproducts that are often produced by the reaction of catalytic material in a catalyst bed (40) which is very thick. For example, when a radial reactor (10) is utilized for the nonoxidative dehydrogenation of ethylbenzene to styrene, the temperature within the catalyst bed (40) drops dramatically as the feed stream passes radially through the catalyst material within the catalyst bed (40). Because other types of reactions are preferentially catalyzed by the catalyst material at lower temperatures, there is an increase in the amount of unwanted by-products that are produced the further the feed stream passes through the catalyst bed (40). By reducing the overall thickness of the active catalyst material within the catalyst bed (40) and by replacing the active catalyst material with inert material, there is a reduction in the overall by-products produced. This reduction in byproducts occurs because there is a reduction in the quantity of the catalyst material that is exposed to the feed stream after the main reaction has occurred. Because the inert material does not react with the components of the feed stream and because the radial reactor is a closed system, there is no drop in the temperature of the feed stream during its passage through the inert material. By reducing the overall thickness of the catalyst bed (40), there is less catalyst that is exposed to the feed stream at a lower temperature than with conventional catalyst beds, thus resulting in a reduction in unwanted byproducts.

The thickness of the active catalyst material layer of the catalyst bed (40) of the invention may vary depending upon the type of reaction that is being catalyzed by the catalyst material. In one preferred embodiment when the catalyst material is utilized for the nonoxidative dehydrogenation of ethylbenzene to styrene, the effective thickness of the catalytic material is from about 4 inches (10 cm) to about 36 inches (90 cm), preferably from about 6 inches (15 cm) to about 24 inches (60 cm), most preferably about 18 inches (45 cm). When the thickness of the catalyst bed (40) in the radial reactor (10) is greater than the desired effective thickness of catalyst material for the catalytic reaction, the remaining space within the catalyst bed (40) is filled with the inert material. The thickness of the ring-shaped, vertical layer (50) of the inert material will thus vary depending upon the overall thickness of the catalyst bed (40) within the radial reactor (10) and with the type of reaction being catalyzed by the catalyst material. Conventional catalyst beds may vary in thickness from as thin as 18 inches (45 cm) or so to 4 feet (120 cm) or more. Thus, the amount of inert material used varies depending upon the amount of catalytic material necessary for an effective reaction. Regardless, any reduction in the amount of the catalyst to the optimum range of four (4) inches (10 cm) to about thirty-six (36) inches (90 cm) is helpful and results in an enhancement in the activity of the catalyst within the catalyst bed.

In one preferred embodiment for the dehydrogenation of ethylbenzene as shown in FIG. 1, the dehydrogenation catalyst contained in the outer, ring-shaped, vertical layer (60) is any conventional commercial or proprietary dehydrogenation catalyst, such as Styromax® catalyst produced by Sud-Chemie Inc., which catalyst material is comprised of iron oxide and potassium oxide. In a particularly preferred embodiment the nonoxidative dehydrogenation catalyst material contained in the outer, ring-shaped, vertical layer (60) is selected from the catalysts disclosed in U.S. Pat. Nos. 6,242,379, 6,191,065 and 6,177,602, which are incorporated herein by reference.

Two or more nonoxidative dehydrogenation catalysts may be utilized together within the active catalyst material layer of the radial reactor (10), each forming a different vertical layer as long as the overall thickness of the layer of the active catalyst material does not dramatically reduce the overall performance of the radial reactor (10). When more than one layer of nonoxidative dehydrogenation catalysts is utilized, preferably at least one of the catalysts has a different performance and/or operating characteristic than at least one of the other catalysts. Different layers of the same catalysts may also be sandwiched around a catalyst with different operating or performance characteristics, depending upon the overall performance or operating characteristics that are desired.

The inert material that is utilized within the catalyst bed (40) is preferably any material which neither promotes catalytic reaction nor interferes with the catalytic reaction of the catalyst material contained in the catalyst bed (40). This inert material also should not react with the components of the feed stream to produce unwanted byproducts. Further, the inert material is preferably formed in a shape which limits the overall pressure drop through the catalyst bed (40). Further, the inert material should have adequate crush strength. The crush strength is preferably the same as or greater than that of the active catalyst material. The inert material also preferably has a similar size to the catalytic material for loading purposes, but can have larger or more numerous openings passing through the individual particles of the inert material to reduce the overall pressure drop.

When the reaction is nonoxidative dehydrogenation reaction, the inert material is preferably a low surface area, for example, alpha alumina, a ceramic material, or a monolithic material.

In one preferred embodiment of a nonoxidative dehydrogenation process of the invention as shown in FIGS. 1, 2 or 3, nonoxidative dehydrogenation catalyst is loaded into the outer, vertical layer (60) and the inert material is loaded in the inner, vertical layer (50) of the catalyst bed (40) of the radial reactor (10), forming separate, vertical layers within the radial reactor (10). In this preferred embodiment, the outer layer (60) of catalyst material is at least four (4) inches (10 cm) thick, preferably from about four (4) inches (10 cm) to about thirty-six (36) inches (90 cm) thick and most preferably from about six (6) inches (15 cm) to about twenty-four (24) inches (60 cm) thick. The remaining material (50) in the catalyst bed (40) is the inert material, preferably a low surface area alpha alumina or ceramic material or a monolithic structure.

After the nonoxidative dehydrogenation catalyst and the inert material are loaded into the catalyst bed (40) of the radial reactor (10), the feed stream, preferably an alkylaromatic and steam, is then passed through the radial reactor (10).

By using this composition of materials for the catalyst bed (40), significant performance advantages are achieved in comparison to the performance of conventional radial reactors with catalyst beds with a thickness greater than about 18 to 36 inches (45-90 cm) which are loaded with only catalyst material. In the inventive design the catalyst material in the outer ring-shaped, vertical layer (60) presents a higher surface area for reaction with the feed stream than if only catalyst material is utilized within the catalyst bed (40) because the overall surface area of the catalyst material portion of the catalyst bed (40) is greater the further you move radially outward from the center (30) of the radial reactor (10) as shown in FIG. 3. In addition, because the volume of the catalyst material which is exposed to the feed stream at the proper operating parameters is optimized, there is a greater “effective” utilization of the catalyst material, thus resulting in higher performance of the catalyst material within the catalyst bed (40). Further, by choosing the right size, shape and strength of the inert material, there is no increase in pressure drop and preferably there is a reduction in the pressure drop as the feed stream passes through the catalyst bed (40). In addition, the effective LHSV is increased. Less unwanted byproducts are also produced because the catalyst material is more effectively utilized. Finally, depending upon the relative cost of the catalyst material versus the inert material, there may be a reduction in the overall cost of a catalyst load within the catalyst bed of the radial reactor.

The principles, preferred embodiments and modes of the operation of the present invention have been described in the foregoing specification. The invention which is protected herein, however, is not to be construed as limited to the particular forms disclosed as theses are to be regarded as illustrative rather than restrictive. Variations and changes may be made by those skilled in the art without departing from the spirit of the invention.

1. A radial reactor for catalytic reaction of gaseous or liquid feed streams comprising
a conventional radial reactor assembly containing a vertical, annular catalyst bed, and
a ring-shaped, vertical layer of material contained within the catalyst bed, wherein the material comprises an active catalyst material contained within a first ring-shaped, vertical layer of the catalyst bed and an inert material contained within a second ring-shaped, vertical layer of the catalyst bed.

2. The radial reactor of claim 1 wherein the active catalyst material is contained within an outer ring-shaped, vertical layer of the catalyst bed and the inert material is contained within an inner ring-shaped, vertical layer of the catalyst bed.

3. The radial reactor of claim 1 wherein the layer containing the catalyst material is at least about 4 inches (10 cm) in thickness, when measured radially from a center of the radial reactor.

4. The radial reactor of claim 1 wherein the layer containing the catalyst material is from about 6 to about 36 inches (15 cm-90 cm) in thickness, when measured radially from a center of the radial reactor.

5. The radial reactor of claim 1 wherein the layer containing the catalyst material is from about 6 to about 24 inches (15 cm-60 cm) in thickness, when measured radially from a center of the radial reactor.

6. The radial reactor of claim 1 wherein the inert material comprises an alpha alumina, ceramic material or a monolithic structure.

7. The radial reactor of claim 1 wherein the overall thickness of the ring-shaped, vertical layer of material contained within the radial reactor is at least about 18 inches (45 cm).

8. The radial reactor of claim 1 wherein the overall thickness of the ring-shaped, vertical layer of material is from about 18 inches (45 cm) to about 48 inches (120 cm).

9. The radial reactor of claim 1 wherein the active catalyst material comprises a plurality of active catalyst products, at least two of which catalyst products have different performance characteristics.

10. A radial reactor for catalytic reaction of gaseous or liquid feed streams comprising
a conventional, radial reactor assembly containing a vertical annular catalyst bed and
a ring-shaped vertical layer of material contained within the catalyst bed, wherein the material comprises an active catalyst material contained within an outer, ring-shaped, vertical layer of the catalyst bed and an inert material contained within an inner, ring-shaped, vertical layer of the catalyst bed.

11. The radial reactor of claim 10 wherein the layer containing the catalyst material is at least about 4 inches (10 cm) in thickness, when measured radially from a center of the radial reactor.

12. The radial reactor of claim 10 wherein the layer containing the catalyst material is from about 6 to about 36 inches (15 cm-90 cm) in thickness, when measured radially from a center of the radial reactor.

13. The radial reactor of claim 10 wherein the inert material comprises an alpha alumina, ceramic material or a monolithic structure.

14. A radial reactor for nonoxidative dehydrogenation of an alkylaromatic feed stream comprising
a conventional radial reactor assembly containing a vertical, annular catalyst bed and
a ring-shaped vertical layer of material contained within the catalyst bed, wherein the material comprises a nonoxidative dehydrogenation catalyst contained within a outer, ring-shaped, vertical layer of the catalytic material and an inert material contained within an inner ring-shaped, vertical layer of the catalyst bed.

15. The radial reactor of claim 14 wherein the layer containing the catalyst material is at least about 4 inches (10 cm) in thickness, when measured radially from a center of the radial reactor.

16. The radial reactor of claim 14 wherein the layer containing the catalyst material is from about 6 to about 36 inches (15 cm-90 cm) in thickness, when measured radially from a center of the radial reactor.

17. The radial reactor of claim 14 wherein the inert material comprises an alpha alumina or ceramic material.

18. A process for the nonoxidative dehydrogenation of an alkylaromatic feed stream comprising
passing an alkylaromatic feed stream through a radial reactor, wherein the radial reactor comprises
a conventional radial reactor assembly containing a vertical, annular catalyst bed, and
a ring-shaped vertical layer of material contained within the catalyst bed, wherein the material comprises an alkylaromatic catalyst material contained within an outer ring-shaped, vertical layer of the catalyst bed and an inert material contained within an inner ring-shaped, vertical layer of the catalyst bed.

19. The process of claim 18 wherein the inert material comprises an alpha alumina or a ceramic material.

20. The process of claim 18 wherein the vertical, annular catalyst bed is from about 18 inches (45 cm) to about 48 inches (120 cm) in thickness.

21. The process of claim 18 wherein the inert material comprises a material which neither promotes catalytic reaction nor substantially interferes with the nonoxidative dehydrogenation of the alkylaromatic feed stream.

22. The process of claim 18 wherein passage of the alkylaromatic feed stream through the radial reactor results in higher selectivity and activity for the catalyst material contained in the catalyst bed than if the material contained in the catalyst bed comprised solely alkylaromatic catalyst material.

23. The process of claim 18 wherein the alkylaromatic catalyst material comprises a plurality of alkylaromatic catalyst products, at least two of which products have different performance characteristics.

24. A process for the nonoxidative dehydrogenation of ethylbenzene comprising
passing an ethylbenzene feed stream through a radial reactor, wherein the radial reactor comprises
a conventional radial reactor assembly containing a vertical, annular catalyst bed, and
a ring-shaped vertical layer of material contained within the catalyst bed, wherein the material comprises an ethylbenzene dehydrogenation catalyst contained within an outer, ring-shaped vertical layer of the catalyst bed and an inert material contained within an inner, ring-shaped vertical layer of the catalyst bed.

25. The process of claim 24 wherein the inert material comprises an alpha alumina, a ceramic material or a monolithic structure.
